(19) United States
(12) Patent Application Publication
(10) Pub. No.: US 2026/0053544 A1
(43) Pub. Date: Feb. 26, 2026

(54) CONDUCTIVE MEMBER TO PROMOTE INSULATIVE MEDIUM FORMATION FOR FACILITATING TISSUE PUNCTURE IN A LIQUID MEDIUM

(71) Applicant: Boston Scientific Scimed, Inc., Maple Grove, MN (US)

(72) Inventors: Christian BALKOVEC, Cambridge (CA); Gareth DAVIES, Toronto (CA)

(21) Appl. No.: 19/305,475

(22) Filed: Aug. 20, 2025

Related U.S. Application Data
(60) Provisional application No. 63/685,645, filed on Aug. 21, 2024.

(51) Int. Cl. A61B 18/14; A61B 18/00; A61B 18/12

(52) U.S. Cl. CPC A61B 18/14; A61B 18/1206; A61B 2018/00083; A61B 2018/00625; A61B 2018/1425; A61B 2018/1435; A61B 2018/144

(57) ABSTRACT

An electrosurgical system for puncturing tissue includes an electrosurgical generator configured to generate radiofrequency (RF) energy and a crossing device connected to the electrosurgical generator. The crossing device includes an electrode positioned at a distal tip of the crossing device, wherein the electrode has a surface texture that is configured to form and hold a gaseous insulation layer that is formed when the crossing device is positioned in an electrically conductive liquid medium and is energized by the electrosurgical generator.

### CROSS REFERENCE TO RELATED APPLICATIONS

This application claims priority to U.S. Provisional Patent Application No. 63/685,645 entitled “CONDUCTIVE MEMBER TO PROMOTE INSULATIVE MEDIUM FORMATION FOR FACILITATING TISSUE PUNCTURE IN A LIQUID MEDIUM,” filed Aug. 21, 2024, which is incorporated herewith in its entirety.

### TECHNICAL FIELD

The present disclosure relates to medical systems and methods for vaporizing tissue in a patient. More specifically, the present disclosure relates to medical systems and methods for puncturing bodily tissues such as the atrial septum with an electrode by dielectric breakdown.

### BACKGROUND

Minimally invasive surgical techniques can employ catheters to provide access into a patient's body. Surgical instruments can be routed through the catheters such that the instruments can be positioned where they are needed for a given procedure. For example, in a cardiac procedure involving the left atrium of the heart, it may be advantageous to route a catheter into the right atrium and perform a transseptal puncture procedure to enter the left atrium. Such a transseptal puncture procedure can be performed using one or more electrodes that are passed through a catheter and energized in the radiofrequency (RF) range. While this procedure can vaporize the targeted septal tissue, it can also vaporize liquids (e.g., blood) that are in contact with the electrode(s).

### SUMMARY

In Example 1, an electrosurgical system for puncturing tissue, the electrosurgical system comprising: an electrosurgical generator configured to generate radiofrequency (RF) energy; and a crossing device connected to the electrosurgical generator, the crossing device including an electrode positioned at a distal tip of the crossing device, wherein the electrode has a surface texture that is configured to form and hold a gaseous insulation layer that is formed when the crossing device is positioned in an electrically conductive liquid medium and is energized by the electrosurgical generator.

In Example 2, the electrosurgical system of Example 1, wherein the texture is formed by the electrode having a groove that is about 0.029 mm.

In Example 3, the electrosurgical system of Example 2, wherein the groove has a helical shape along a length of the electrode.

In Example 4, the electrosurgical system of Example 3, wherein the electrode comprises a helical coil that defines the groove.

In Example 5, the electrosurgical system of Example 4, wherein the helical coil is wrapped around a central elongate member.

In Example 6, the electrosurgical system of Example 5, wherein a diameter of the helical coil is between one fourteenth and one tenth of a diameter of the central elongate member.

In Example 7, the electrosurgical system of any of Examples 1-6, wherein the central elongate member includes an exterior electrically insulating layer extending proximal from the electrode.

In Example 8, the electrosurgical system of any of Examples 2-7, wherein the groove is one of an array of grooves that form a corrugations along a length of the electrode.

In Example 9, the electrosurgical system of any of Examples 1-8, wherein the texture is formed by the electrode having an arithmetic average roughness profile (Ra) between about 52 to about 64.

In Example 10, the electrosurgical system of any of Examples 1-9, wherein the texture is formed in a crisscross pattern.

In Example 11, the electrosurgical system of any of Examples 1-10, wherein the liquid medium is blood.

In Example 12, the electrosurgical system of any of Examples 1-11, further comprising a dilator positioned near a distal end of the crossing device, proximate from the electrode.

In Example 13, the electrosurgical system of any of Examples 1-12, wherein the crossing device is a wire.

In Example 14, the electrosurgical system of Example 13 and any of claims 1-12, further comprising an electrically insulative layer extending over the wire except at the electrode.

In Example 15, the electrosurgical system of any of Examples 1-14, wherein the crossing device is a needle.

In Example 16, an electrosurgical system for puncturing tissue, the electrosurgical system comprising: an electrosurgical generator configured to generate radiofrequency (RF) energy; and a crossing device connected to the electrosurgical generator, the crossing device including an electrode positioned at a distal tip of the crossing device, wherein the electrode has a surface texture that is configured to form and hold a gaseous insulation layer that is formed when the crossing device is positioned in an electrically conductive liquid medium and is energized by the electrosurgical generator.

In Example 17, the electrosurgical system of Example 16, wherein the texture is formed by the electrode having a groove that is about 0.029 mm.

In Example 18, the electrosurgical system of Example 17, wherein the groove has a helical shape along a length of the electrode.

In Example 19, the electrosurgical system of Example 18, wherein the electrode comprises a helical coil that defines the groove.

In Example 20, the electrosurgical system of Example 17, wherein the groove is one of an array of grooves that form a corrugations along a length of the electrode.

In Example 21, the electrosurgical system of Example 16, wherein the texture is formed by the electrode having an arithmetic average roughness profile (Ra) between about 52 to about 64.

In Example 22, the electrosurgical system of Example 16, wherein the texture is formed in a crisscross pattern.

In Example 23, the electrosurgical system of Example 16, wherein the liquid medium is blood.

In Example 24, the electrosurgical system of Example 16, further comprising a dilator positioned near a distal end of the crossing device, proximate from the electrode.

In Example 25, the electrosurgical system of Example 16, wherein the crossing device is a wire.

In Example 26, the electrosurgical system of Example 25, further comprising an electrically insulative layer extending over the wire except at the electrode.

In Example 27, the electrosurgical system of Example 16, wherein the crossing device is a needle.

In Example 28, an electrosurgical system for puncturing tissue, the electrosurgical system comprising: an electrosurgical generator configured to generate radiofrequency (RF) energy; and a crossing device connected to the electrosurgical generator, the crossing device including an electrode positioned at a distal tip of the crossing device, wherein the electrode has a helical coil that is configured to form and hold a gaseous insulation layer that is formed when the crossing device is positioned in an electrically conductive liquid medium and is energized by the electrosurgical generator.

In Example 29, the electrosurgical system of Example 28, wherein the helical coil is wrapped around a central elongate member.

In Example 30, the electrosurgical system of Example 29, wherein a diameter of the helical coil is between one fourteenth and one tenth of a diameter of the central elongate member.

In Example 31, the electrosurgical system of Example 30, wherein the central elongate member includes an exterior electrically insulating layer extending proximal from the electrode.

In Example 32, the electrosurgical system of Example 28, wherein the liquid medium is blood.

A method of operating an electrosurgical system, the method comprising: positioning a crossing member, wherein the crossing member includes a crossing device; energizing an electrode of the crossing device using an electrosurgical generator; forming and coalescing gaseous bubbles on the electrode; and vaporizing tissue with the energized electrode.

In Example 34, the method of Example 33, wherein the crossing member is positioned near to the tissue but not in contact with the tissue prior to energizing the electrode.

In Example 35, the method of Example 33, wherein the crossing member is positioned in contact with the tissue prior to energizing the electrode.

While multiple embodiments are disclosed, still other embodiments of the present disclosure will become apparent to those skilled in the art from the following detailed description, which shows and describes illustrative embodiments of the disclosure. Accordingly, the drawings and detailed description are to be regarded as illustrative in nature and not restrictive.

### BRIEF DESCRIPTION OF THE DRAWINGS

FIG. 1 is a schematic diagram illustrating an example electrosurgical system for treating a patient, such as a heart or the vasculature of a patient, including an electrosurgical generator and a transseptal crossing system, consistent with various aspects of the present disclosure.

FIG. 2 is a cutaway view of a heart including an atrial septum and the transseptal crossing system, consistent with various aspects of the present disclosure.

FIG. 3 is a side view of a distal end of the transseptal crossing system, consistent with various aspects of the present disclosure.

FIG. 4 is a side view of a distal tip of the transseptal crossing system, consistent with various aspects of the present disclosure.

FIG. 5 is a side view of an alternative distal tip of the transseptal crossing system, consistent with various aspects of the present disclosure.

FIG. 6 is a side cross-sectional view of an alternative distal tip of the transseptal crossing system, consistent with various aspects of the present disclosure.

FIG. 7 is a flowchart of a method of crossing the atrial septum, consistent with various aspects of the present disclosure.

FIGS. 8A and 8B are a series of schematic diagrams of operations of crossing the atrial septum according to the method of FIG. 7, consistent with various aspects of the present disclosure.

FIG. 9 is a flowchart of a method of operating the electrosurgical system to puncture tissue, consistent with various aspects of the present disclosure.

FIGS. 10A-10F are a series of schematic diagrams of operations of puncturing tissue according to the method of FIG. 9, consistent with various aspects of the present disclosure.

FIG. 11 is a flowchart of an alternative method of operating an alternative electrosurgical system to puncture and slice tissue, consistent with various aspects of the present disclosure.

While the disclosure is amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure as defined by the appended claims.

### DETAILED DESCRIPTION

For purposes of promoting an understanding of the principles of the present disclosure, reference is now made to the examples illustrated in the drawings, which are described below. The illustrated examples disclosed herein are not intended to be exhaustive or to limit the disclosure to the precise form disclosed in the following detailed description. Rather, these exemplary embodiments were chosen and described so that others skilled in the art may use their teachings. It is not beyond the scope of this disclosure to have a number (e.g., all) the features in a given example used across all examples. Thus, no one figure should be interpreted as having any dependency or requirement related to any single component or combination of components illustrated therein. Additionally, various components depicted in a given figure may be, in examples, integrated with various ones of the other components depicted therein (and/or components not illustrated), all of which are considered to be within the ambit of the present disclosure.

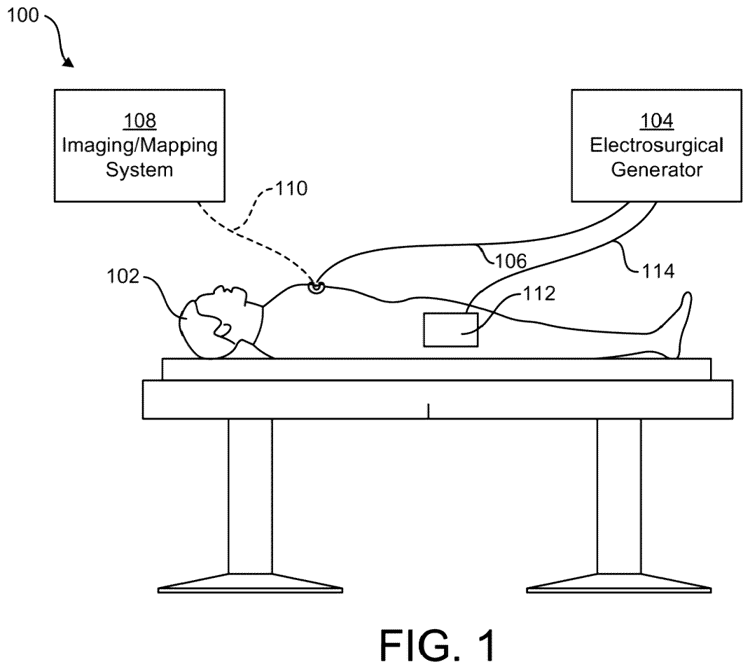

FIG. 1 shows an electrosurgical system 100 for treating a patient 102. In the illustrated embodiment, the system 100 includes an electrosurgical generator 104 with a transseptal crossing system 106 and an imaging/mapping system 108 for tracking the crossing system 106 in the patient 102. The imaging/mapping system 108 can use an external fluoroscopy system (not shown) and/or a mapping catheter 110 (shown in phantom) (such as, for example, the OPAL HDx™ mapping system from the Boston Scientific Corporation).

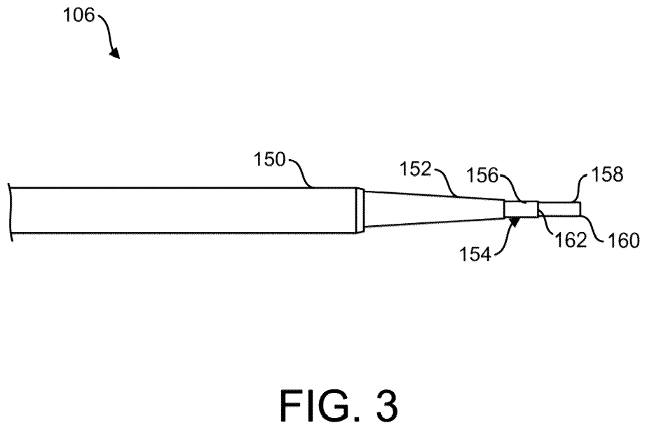

In the illustrated embodiment, the electrosurgical generator 104 is configured to provide energy, such as radiofrequency (RF) electrical energy, to the crossing system 106. Typically, the conductive crossing system 106 is electrically insulated with the exception of a small distal portion, formed as a vaporizing electrode (shown in FIG. 3), that is intended to deliver the RF energy to the target tissue. To achieve tissue vaporization, the target tissue is rapidly heated. If heating is too slow, the tissue is desiccated rather than vaporized. Rapid heating is achieved through high current density at the electrode-tissue interface, meaning the electrode is either relatively small and/or the energy delivered is relatively high.

The delivery of energy to the target tissue can also heat the crossing system 106 itself. Thus, the electrical insulation around the crossing system 106 (especially near the electrode) should be able to withstand such heat without breaking down. While typically the insulation has been made from per- and polyfluoroalkyl substances (PFAS) (e.g., polytetrafluoroethylene (PTFE)), other materials may have the advantage of being less environmentally problematic. Such alternative materials, however, may lack the heat performance of the traditional materials, so the crossing system 106 should be designed to reduce the heat generation within itself while still providing adequate heat generation in the target tissue.

In the illustrated embodiment of FIG. 1, the crossing system 106 is monopolar and includes a single vaporizing electrode (i.e., an active electrode), so the system 100 also includes a patch electrode 112 (i.e., an indifferent or dispersive electrode). The patch electrode 112 has a large surface area to lower current density, so the patch electrode 112 is typically located on the back, buttocks, or upper leg of the patient 102, although there may be other suitable locations as well. The patch electrode 112 returns the RF electrical energy to the generator 104 through the lead 114. In some embodiments, the RF energy for a monopolar puncture function is provided by the electrosurgical generator 104 at a selected voltage and a continuous current (100% on, or 100% duty cycle). For example, if a power setting of 50 watts (W) is used for puncturing (which can mean that the instantaneous power is higher than 50 W), the voltage can range from approximately 164 volts (V) to 400 V root mean square (RMS).

In addition, the electrosurgical generator 104 can include a plurality of functions and provide programmed and custom settings via an interface (not shown). For example, the electrosurgical generator 104 provides RF energy to the crossing system 106 as an alternating current having a frequency in the range of 100 kilohertz (kHz) to 10 megahertz (MHz). Such puncturing RF energy can be applied in the form of a continuous waveform signal or in bursts of a waveform signal. In the latter case, the individual bursts of the waveform signal can have a duration of about 300 milliseconds (ms) with a rest interval between pulses of about 700 ms, although other durations of bursts and intervals can be used. In some embodiments, the waveform signals themselves can be sinusoidal or square waves that are bi-phasic. Furthermore, the electrosurgical generator 104 can be couplable to other electrosurgical tools and/or the electrosurgical generator 104 can receive signals (e.g., from the crossing system 106) to monitor the patient 102.

The components and configuration of electrosurgical system 100 allow for target tissue to be vaporized. In some embodiments, the tissue vaporization allows the crossing system 106 to puncture through the atrial septum for treatment of the left side of the heart of the patient 102. While examples of the devices, systems, and methods of the present disclosure are presented in the context of a transeptal puncture, a person having ordinary skill in the art will recognize other applicable contexts. For example, the electrosurgical systems of the present disclosure can be employed to puncture a pericardium layer of a patient for epicardial access and/or to remove accumulation of atheromatous material on the inner walls of vascular lumens.

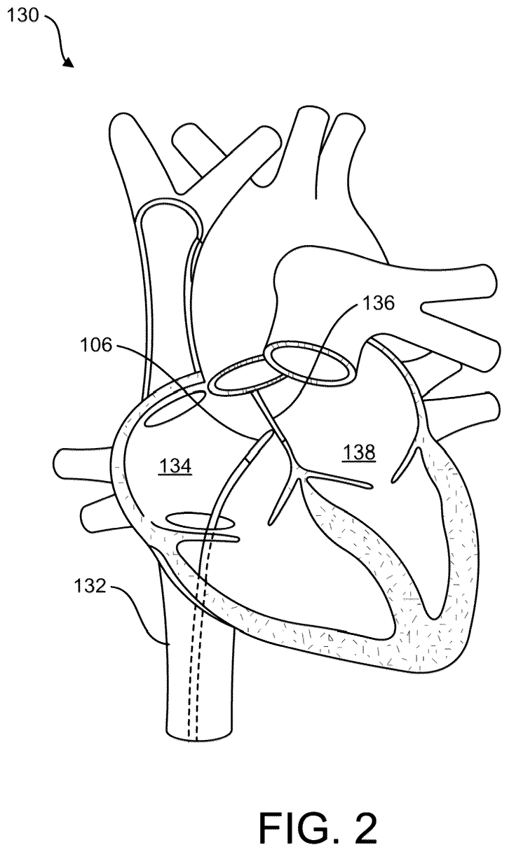

FIG. 2 shows a heart 130 of the patient 102 (shown in FIG. 1) with selected portions cut away. In the illustrated embodiment, the crossing system 106 extends through the inferior vena cava 132 from a surgical entry site (not shown) that is distal to the heart 130. The distal end of the crossing system 106 is positioned in the right atrium 134 and is in contact with the atrial septum 136. In the illustrated embodiment, the tip of the crossing system 106 is positioned at the fossa ovalis since this region of the atrial septum 136 is relatively safe and easy to puncture. Once the atrial septum 136 is crossed, the physician will have access to the left atrium 138 (e.g., for treatment thereof).

In some use cases, such as a septal crossing, the electrode will be surrounded by a conductive liquid medium, such as blood, that is near the target tissue. When the crossing system 106 is operating in such an environment, any surface of the electrode that is in contact with the conductive liquid (instead of with the target tissue) provides a shunt path for the electrical current. These alternative electrical pathways do not help vaporize the target tissue, so they decrease the efficiency of the tissue vaporization process. Furthermore, these pathways can cause the blood to locally coagulate and form thrombotic material that can lead to embolization and subsequent deleterious health effects for the patient 102 (shown in FIG. 1). Therefore, components and methods to prevent such occurrences are discussed in the present disclosure.

FIG. 3 shows a distal end of the crossing system 106. In the illustrated embodiment, the crossing system 106 includes a sheath 150, a dilator 152, and a crossing device 154. The crossing device 154 is an elongated tissue vaporization device that can have the form of, for example, a wire, a needle, forceps, scalpels, or other devices that puncture and/or cut tissue. In some embodiments, a wire is a solid, stiff but elastically deformable member with a generally straight and/or helical configuration. In some embodiments, a needle is a hollow, flexible member with a generally straight configuration through which fluid can be pumped. The fluid can exit near an electrode that is positioned at the distal end of the needle, and the electrode can be connected to the electrosurgical generator 104 with a conductor since the flexible member can be made from an electrically insulating material. In some embodiments, forceps are a dual-levered instrument capable of grasping and/or holding tissue or other objects between their distal ends. In some embodiments, a scalpel is a bladed instrument with a sharpened edge capable of cutting tissue or other objects.

In some embodiments, the crossing system 106 has an overall length between about 55 centimeters (cm) and about 300 cm. The sheath 150 is an elongate member with a central lumen (not shown), in which the dilator 152 and the crossing device 154 are slidably positioned. The central lumen diameter is similar to the outer diameter of the majority of the dilator 152 (except for the distal tip), and the sheath 150 is tapered at the distal end to make the transition between the sheath 150 and the dilator 152 smoother. In addition, the sheath 150 can be a steerable sheath and/or have a fixed or adjustable curve at the distal end for positioning of the dilator 152 and the crossing device 154. In some embodiments, the sheath 150 and the dilator 152 are generally similar to those of the VersaCross™ Access Solution from Boston Scientific.

In the illustrated embodiment, the dilator 152 is an elongate member with a central lumen (not shown), in which the crossing device 154 is slidably positioned. The central lumen diameter is similar to the outer diameter of the crossing device 154, and the dilator 152 is tapered at the distal end to make the transition between the dilator 152 and the crossing device 154 smoother. In addition, the dilator 152 can have a fixed or adjustable curve at the distal end for positioning of the crossing device 154 against the atrial septum 136 (shown in FIG. 2).

In the illustrated embodiment, the crossing device 154 is an elongate member with a central electrical conductor (not shown) (e.g., comprising stainless steel, nitinol, platinum, gold, iridium, or combinations thereof) that is surrounded by an electrically insulative layer 156 (e.g., comprising parylene, polyimide, polyethylene terephthalate (PET), polyurethane, silicon dioxide (SiO₂), PTFE heat shrink, or combinations thereof. In some embodiments, a diameter of the crossing device 154 is between about 0.50 millimeters (mm) and about 1.0 mm. An electrode 158 forms the distal end of the crossing device 154, and the electrode 158 is electrically connected to the electrosurgical generator 104 (shown in FIG. 1) via the central electrical conductor. The electrode 158 is an electrical conductor comprising, for example, stainless steel, nitinol, gold, platinum, tungsten, iridium, or alloys thereof. In some embodiments, the electrode 158 is an exposed section of the central electrical conductor, and in other embodiments, the electrode 158 is connected to the distal end of the central electrical conductor and has the same diameter as the insulative layer 156. The electrode 158 has a cylindrical shape with a flat distal end having a distal circular edge 160 and a proximal circular edge 160 where the proximal end of the electrode 158 and the distal end of the insulative layer 156 are coterminous. Thus, the electrode 158 is atraumatic (i.e., not sharp) and has distinct circular edges 160, 162 at the distal and proximal ends, respectively. In other embodiments, however, the crossing device has a distal domed shape.

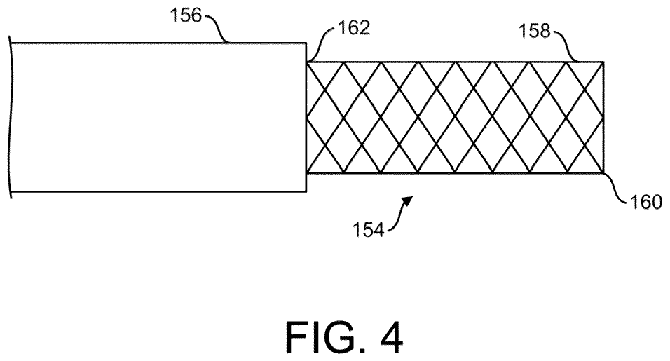

FIG. 4 shows an embodiment of the crossing device 154 wherein the electrode 158 has a roughened surface texture that includes, for example, irregularities, etching, deformations, notches, or other features that result in a non-uniform and/or non-smooth surface. In some embodiments, the surface texture has an arithmetic average roughness profile (Ra) between about 52 to about 64, between about 55 to about 61, or about 58. The surface texture of the electrode 158 can be localized to just the electrode 158 or the surface texture can extend proximally along some or all of the central electrical conductor (not shown). The surface texture of the electrode 158 is on the circumferential side, and in some embodiments, the surface texture can extend across the distal face of the electrode 158. The surface texture of the electrode 158 can be formed using any suitable method, for example, by removing material (e.g., using abrasive material (e.g., 240 grit sandpaper) or a chemical etching process) or by displacing/deforming material (e.g., using a knurling tool). Accordingly, the topography of the surface texture of the electrode 158 can be random or it can be orderly with an orientation (e.g., longitudinal, circumferential, helical, or crisscross).

In some embodiments, the diameter of the electrode 158 is about 0.50 millimeters (mm) to about 1.0 mm. In some embodiments, the length of the electrode 158 is about 0.50 mm to about 1.5 mm. In some embodiments, the exposed surface area of the electrode 158 is about 1.0 mm² to about 3.5 mm².

The rough surface texture of the electrode 158 provides irregularities that increase local electrical current density. Thus, the irregularities serve as nucleation sites for gaseous bubbles due to the local heating thereat. Furthermore, the irregularities allow the gaseous bubbles to cling to the electrode 158 in a Wenzel state, as will be explained below. Furthermore, the distal and proximal circular edges 160, 162 of the electrode 158 also provide nucleation sites for gaseous bubbles due to the locally increased current density and heating. These features of the present disclosure are in contrast with traditional transseptal crossing electrodes that have smooth and/or polished finishes that are created, for example, by melting the material when forming rounded/spherical distal ends.

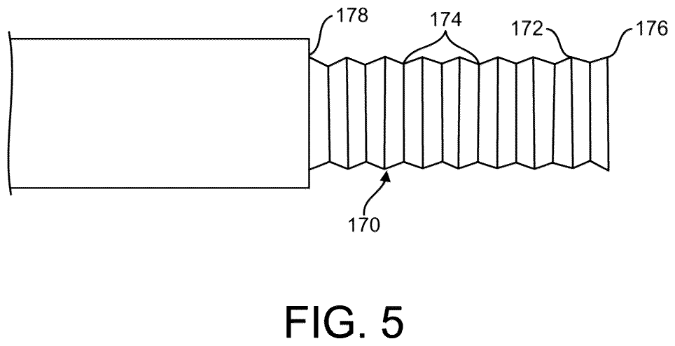

FIG. 5 shows an alternative embodiment of a crossing device 170 wherein an electrode 172 has a corrugated surface texture. In the illustrated embodiment, the corrugations give the electrode 172 an accordion-like shape due to the array of grooves 174 formed in the electrode 172. The average diameter of the electrode 158 is about 0.62 mm and the length of the electrode is about 1.5 mm, the corrugations have a wavelength of about 0.058 mm and an amplitude of about 0.029 mm. The surface texture of the electrode 172 can be localized to just the electrode 172 or it can extend proximally along some or all of the central electrical conductor (not shown). The surface texture of the electrode 172 can be formed using any suitable method, for example, by removing material (e.g., using a cutting tool) or by displacing material (e.g., a crimping or rolling die). In other embodiments, the surface texture is oriented in other directions, such as, for example, longitudinally or helically. In the former such embodiment, the electrode would have a splined shape due to the array of grooves being oriented longitudinally. In the latter such embodiment, the electrode would have a threaded shape with a single helical groove that winds around and along the length of the electrode 172.

Regardless of the orientation, the peak(s) of the surface texture serve as nucleation sites for gaseous bubbles due to the locally increased current density and heating. The surface texture also allows the gaseous bubbles to cling to the electrode 172 in a Wenzel state in the groove(s) (e.g., grooves 174). Furthermore, the distal and proximal circular edges 176, 178 of the electrode 172 also provide nucleation sites for gaseous bubbles, respectively.

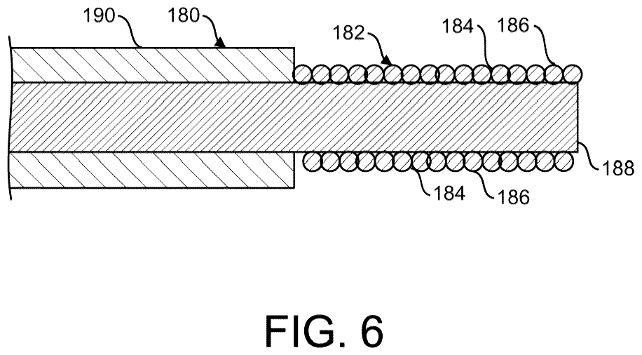

FIG. 6 shows an alternative embodiment of a crossing device 180 wherein an electrode 182 has a single helical groove 184 that winds around the length of the electrode 182. The groove 184 is defined by a helical coil 186, and the coil 186 is wrapped around an elongate central electrical conductor 188. The diameter of the coil 186 is about 0.058 mm, and the diameter of the conductor 188 is about 0.50 mm. So, the diameter of the coil 186 is between about one fourteenth and about one tenth of the diameter of the conductor 188 or about one twelfth of the diameter of the conductor 188. In some embodiments, the coil 186 is welded, soldered, brazed, adhered, or otherwise affixed to the conductor 188 such that the coil 186 can transmit electricity from the conductor 188 to the patient 102 (shown in FIG. 1). The coil 186 can comprise the same or different material from that of the conductor 188. In some alternate embodiments, the coil 186 extends along a length of the conductor 188 such that the majority of the coil 186 is covered by an electrically insulating layer 190. Thus, the peak of the surface texture (i.e., the outermost portion of the coil 186) provide nucleation sites for gaseous bubbles due to the locally increased current density and heating and allow the gaseous bubbles to cling to the electrode 182 in a Wenzel state in the groove 182.

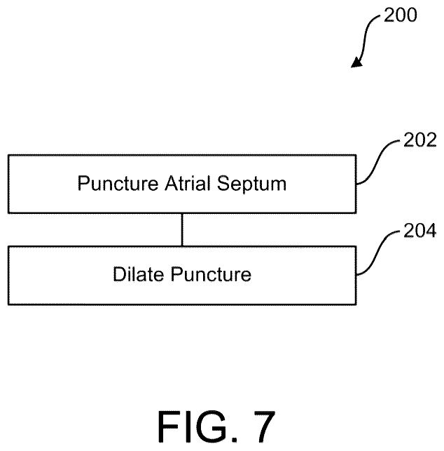

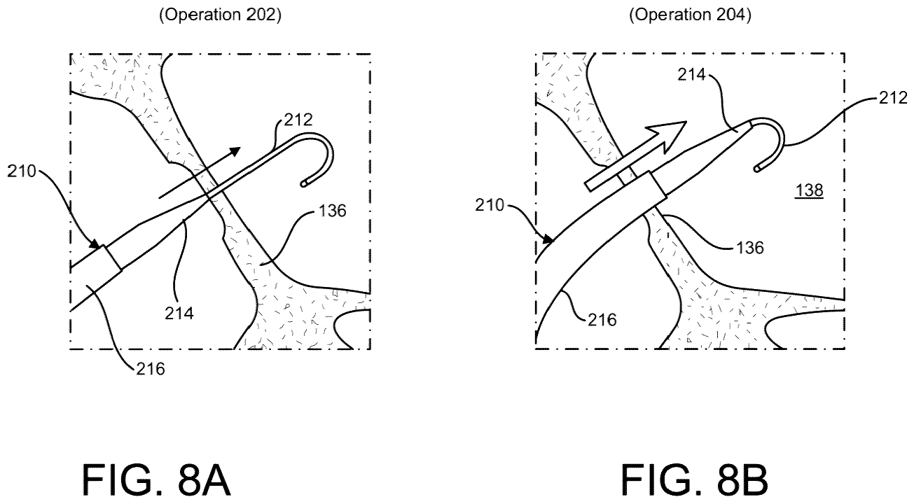

FIG. 7 shows a method 200 of crossing the atrial septum 136 using the crossing system 210 which can be the same as or similar to the crossing system 106 (shown in FIG. 2). FIGS. 8A and 8B show the operations of crossing the atrial septum 136. The distal tip of the crossing system 210 can have any of the embodiments of the crossing device of the present disclosure (e.g., crossing device 154, 170, or 180). FIGS. 7 and 8A and 8B will now be discussed in conjunction with one another, and each operation of the method 200 is illustrated by a corresponding one of FIGS. 8A and 8B.

In the illustrated embodiment, the method 200 begins with a crossing system 210 being positioned near the atrial septum 136. At operation 202, as shown in FIG. 8A, the crossing device 212 is energized by the electrosurgical generator 104, and the physician applies distally-oriented force to the crossing device 212 (as indicated by the arrow). Thus, the crossing device 212 punctures through the atrial septum 136, and the crossing device 212 is deenergized. As shown in FIG. 8A, the crossing device 212 has a J-shape that is elastically deformed to be straight when the crossing device 212 is in the dilator 214, so the crossing device 212 returns to the J-shape when the crossing device 212 exits the dilator 214.

At operation 204, as shown in FIG. 8B, the physician applies distally-oriented force to the dilator 214 and to the sheath 216 (as indicated by the arrow). Thus, the dilator 214 and the sheath 216 follow the crossing device 212 through the atrial septum 136. As the dilator 214 follows the crossing device 212, the puncture in the atrial septum 136 is gently stretched by the dilator 214 so that the sheath 216 can enter the left atrium 138. Once the sheath 216 is positioned in the left atrium 138, one or more of the crossing device 212, the dilator 214, and the sheath 216 can be withdrawn and replaced with another component, such as, for example, a left atrial appendage closure (LAAC) implant device (not shown).

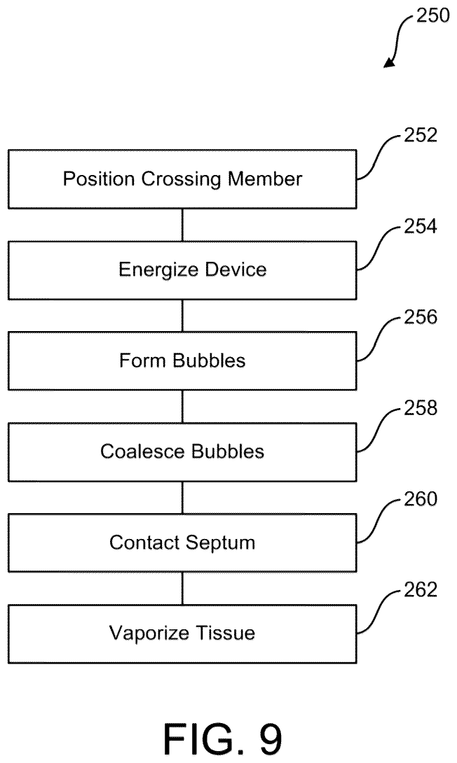

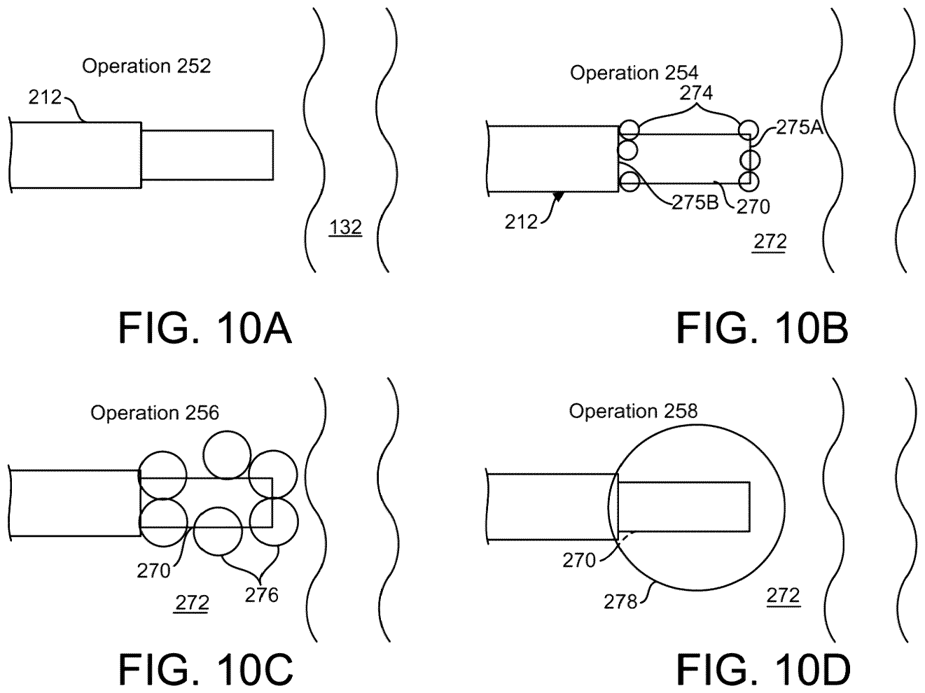

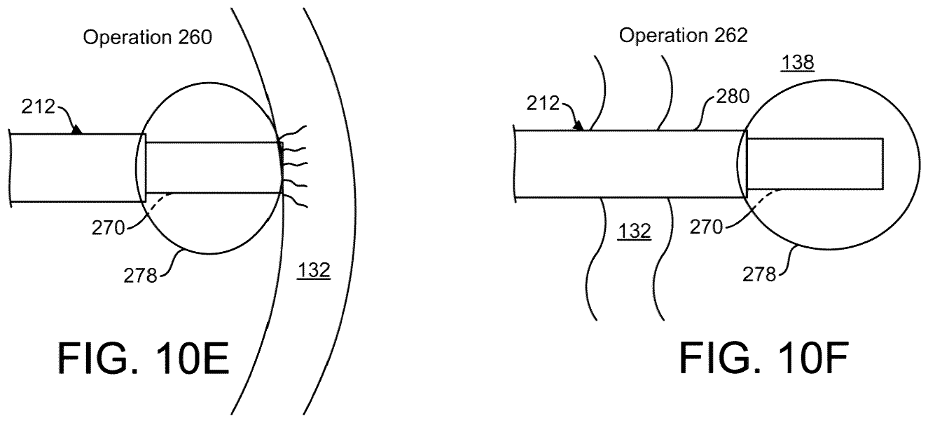

FIG. 9 shows a method 250 of puncturing the atrial septum 136. FIGS. 10A-10F show the operations of puncturing the atrial septum 136 using the crossing device 212. FIGS. 9 and 10A-10F will now be discussed in conjunction with one another, and each operation of the method 250 is illustrated by a corresponding one of FIGS. 10A-10F.

In the illustrated embodiment, the method 250 begins with operation 252. As shown in FIG. 10A, the crossing device 212 is positioned near the atrial septum 136.

At operation 254, as shown in FIG. 10B, the crossing device 212 is energized, which also energizes the electrode 270. The electrode 270 can be any of the embodiments of the electrode of the present disclosure (e.g., electrode 158, 172, or 182). Energizing of the electrode 270 passes RF energy into the blood 272 that surrounds the electrode 270 since the blood 272 is electrically conductive. This locally gasifies the blood 272 and forms small gaseous bubbles 274 that stick to the electrode 270, especially around the major nucleation sites. More specifically, these major sites are along the distal and proximal circular edges 275A-275B of the electrode 270 (i.e., the circumferences of the distal and proximal ends of the electrode 270, respectively).

At operation 256, as shown in FIG. 10C, the electrode 270 is still energized and continues to generate heat and locally gasify the blood 272. The continued gasification rapidly increases the size of the small gaseous bubbles 274 (shown in FIG. 10B) to medium gaseous bubbles 276. There are also new small gaseous bubbles (not shown) that are grown on exterior areas of the electrode 270 that are spaced apart from the major nucleation sites. These new small gaseous bubbles grow on the many minor nucleation sites along the surface texture of the electrode 270 that are provided by the rough and/or grooved surface of the electrode 270, and said small gaseous bubbles also grow into the medium gaseous bubbles 276. Furthermore, the medium gaseous bubbles 276 are in a Wenzel state on the electrode 270. The Wenzel state increases adhesion energy compared to a surface that is substantially flat/smooth, meaning that the medium gaseous bubbles 276 adhere more readily to the rough and/or grooved surface texture of the electrode 270 than they would to a prior art electrode.

At operation 258, as shown in FIG. 10D, the electrode 270 is still energized and continues to locally gasify the blood 272. The medium gaseous bubbles 276 (shown in FIG. 10C) grow and are retained on the electrode 270 and eventually coalesce into a gaseous insulation layer 278. The gaseous insulation layer 278 is a single large gaseous bubble that surrounds substantially the entirety of the electrode 270. While voltage is still being applied to the electrode 270 at operation 258, the current flow through the electrode 270 is greatly reduced compared to that of operations 254 and 256 since the electrode 270 is electrically insulated from the blood 272. The great reduction in current flow means that less heat is produced, and less (if any) of the blood 272 is being locally gasified. Thus, the gaseous insulation layer 278 can maintain its size and adherence to the electrode 270.

At operation 260, as shown in FIG. 10E, the crossing device 212 is advanced by the physician so that the distal end of the electrode 270 contacts the atrial septum 136. The gaseous insulation layer 278 is deformed as the distance between the electrode 270 and the atrial septum 136 closes. Eventually, the thickness of the gaseous insulation layer 278 between the electrode 270 and the atrial septum 136 is no longer sufficient, which causes a local dielectric breakdown of the gaseous insulation layer 278. Thereby, the distal end of the electrode 270 and the atrial septum 136 are electrically connected, and virtually all of the RF energy being transmitted to the electrode 270 flows into the cells of the atrial septum 136 that are closest to the electrode 270, vaporizing the cells.

At operation 262, as shown in FIG. 10F, the physician has maintained distally-oriented force on the crossing device 212 as the cells at the end of the electrode 270 have been vaporized. As these cells are vaporized, the crossing device 212 penetrates through the atrial septum 136 with minimal tenting of the atrial septum 136 and with minimal jumping of the crossing device 212 upon breakthrough. The ease of the puncture means that the physician does not need to exert as much force on the crossing device 212 compared to a prior art crossing system that has inefficient or diffuse RF energy delivery. The inefficiency or diffuseness of the prior art can result from a flat, smooth, and/or polished prior art electrode that does not create and/or does not retain its gaseous bubbles. Such an electrode would maintain contact with the blood 272 even when in contact with the atrial septum 136, so only a fraction of the RF energy provided to the electrode would be delivered to the atrial septum 136. The inefficient or diffuse delivery of the RF energy can result in the tissue cells receiving an insufficient amount of power for vaporization but enough for the tissue cells to be desiccated instead. Desiccation increases the electrical resistance of the affected tissue cells because there are fewer polar water molecules in the cells to conduct electricity, so the result is that even more power flows into the blood 272 instead of into the atrial septum 136.

In some embodiments, the time to puncture the atrial septum 136 can be about 300 ms to about 400 ms. However, some electrosurgical generators will continue to deliver power to the electrode 270 due to their programming and/or user selection/input. The total “on” time of such a system can be, for example, multiple seconds(s) (e.g., 2 s to 5 s), which means that the electrode 270 is still powered well after the puncture has been completed. In such a scenario, the gaseous insulation layer 278 may have been wiped off of the electrode 270 as it passed through the atrial septum 136. But the gaseous insulation layer 278 can reform in the left atrium, for example, in a similar manner to that of operations 254-258. Once the gaseous insulation layer 278 is reformed, the amount of current flowing through the electrode 270 will again be reduced until cessation of power delivery by the electrosurgical generator 104 (shown in FIG. 1). On the other hand, a prior art electrode that does not retain its gaseous bubbles, after puncturing the atrial septum 136, would be generating and releasing gaseous bubbles in the left atrium 138 of the heart 130 (shown in FIG. 2). Such free-floating bubbles can be dangerous to the patient 102 (shown in FIG. 1) for a variety of reasons.

The components and configuration of the crossing device 212 and the creation of the gaseous insulation layer 278 around the electrode 270 requires less electrical power than a prior art system with a smooth, flat, and/or polished electrode that does not retain its gaseous bubbles. Less power flowing through the electrode 270 means that there is less heat generated at the electrode 270. Thus, there is less heat transferring to the electrically insulative layer 280, allowing for a material to be used for the electrically insulative layer 280 that is less heat resistant than in prior art crossing devices.

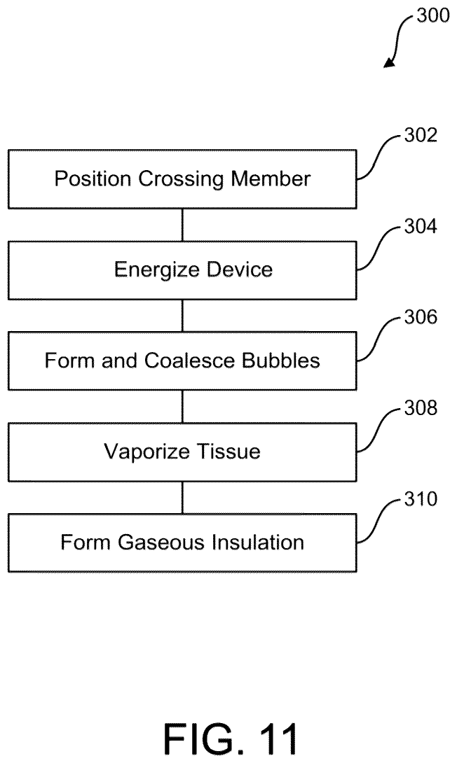

FIG. 11 shows a method 300 of puncturing the atrial septum 136. The method 300 is similar to that of the method 250 albeit with distinct differences. For example, the insulative gaseous layer is formed after the electrode makes contact with the atrial septum instead of before.

In the illustrated embodiment, the method 300 begins with operation 302 wherein the crossing device is positioned in contact with the atrial septum.

At operation 304, the crossing device is energized, which also energizes the electrode. The electrode can be any of the embodiments of the electrodes of the present disclosure, respectively. Energizing of the crossing device passes RF energy into the blood in and around the electrode as well as into the atrial septum. Due to the diffuse distribution of RF energy, the cells in the atrial septum are not vaporized yet. The blood, however, is still locally gasified at the surface of the electrode to form small gaseous bubbles at the major nucleation sites.

At operation 306, the crossing device is still energized and the electrode continues to generate heat and locally gasify the blood. This gasifying of the blood rapidly increases the size of the small gaseous bubbles, which are retained on the electrode and eventually coalesce into a single large gaseous bubble. Such a large gaseous bubble is positioned between the electrode and the blood, although not between the distal end of the electrode and the atrial septum since those two were already in direct contact with one another.

At operation 308, the crossing device is still energized, and virtually all of the RF energy being transmitted to the electrode now flows into the cells of the atrial septum. This energy vaporizes the cells of the atrial septum that are closest to (e.g., in direct contact with) the electrode.

At operation 310, the physician has maintained distally-oriented force on the crossing device as the cells at the end of the electrode have been vaporized. This vaporization allows the crossing device to puncture through the atrial septum. In doing so, a portion of the large gaseous bubble that existed outside of the electrode may have been wiped off of the electrode as it passed through the atrial septum. But a gaseous insulation layer can be formed on the electrode in the left atrium, for example, in a similar manner to that of operation 262 of the method 250 (shown in FIG. 9). Once the gaseous insulation layer is formed, the amount of current flowing through the electrode will be greatly reduced until cessation of power delivery by the electrosurgical generator 104 (shown in FIG. 1).

It is well understood that methods that include one or more steps, the order listed is not a limitation of the claim unless there are explicit or implicit statements to the contrary in the specification or claim itself. It is also well settled that the illustrated methods are just some examples of many examples disclosed, and certain steps may be added or omitted without departing from the scope of this disclosure. Such steps may include incorporating devices, systems, or methods or components thereof as well as what is well understood, routine, and conventional in the art.

The connecting lines shown in the various figures contained herein are intended to represent exemplary functional relationships and/or physical couplings between the various elements. It should be noted that many alternative or additional functional relationships or physical connections may be present in a practical system. However, the benefits, advantages, solutions to problems, and any elements that may cause any benefit, advantage, or solution to occur or become more pronounced are not to be construed as critical, required, or essential features or elements. The scope is accordingly to be limited by nothing other than the appended claims, in which reference to an element in the singular is not intended to mean “one and only one” unless explicitly so stated, but rather “one or more.” Moreover, where a phrase similar to “at least one of A, B, or C” is used in the claims, it is intended that the phrase be interpreted to mean that A alone may be present in an embodiment, B alone may be present in an embodiment, C alone may be present in an embodiment, or that any combination of the elements A, B or C may be present in a single embodiment; for example, A and B, A and C, B and C, or A and B and C. The terms “couples,” “coupled,” “connected,” “attached,” and the like along with variations thereof are used to include both arrangements wherein two or more components are in direct physical contact and arrangements wherein the two or more components are not in direct contact with each other (e.g., the components are “coupled” via at least a third component), but still cooperate or interact with each other.

In the detailed description herein, references to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art with the benefit of the present disclosure to affect such feature, structure, or characteristic in connection with other embodiments whether or not explicitly described. After reading the description, it will be apparent to one skilled in the relevant art(s) how to implement the disclosure in alternative embodiments.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

We claim:

1. An electrosurgical system for puncturing tissue, the electrosurgical system comprising:
an electrosurgical generator configured to generate radiofrequency (RF) energy; and
a crossing device connected to the electrosurgical generator, the crossing device including an electrode positioned at a distal tip of the crossing device, wherein the electrode has a surface texture that is configured to form and hold a gaseous insulation layer that is formed when the crossing device is positioned in an electrically conductive liquid medium and is energized by the electrosurgical generator.

2. The electrosurgical system of claim 1, wherein the texture is formed by the electrode having a groove that is about 0.029 mm.

3. The electrosurgical system of claim 2, wherein the groove has a helical shape along a length of the electrode.

4. The electrosurgical system of claim 3, wherein the electrode comprises a helical coil that defines the groove.

5. The electrosurgical system of claim 2, wherein the groove is one of an array of grooves that form a corrugations along a length of the electrode.

6. The electrosurgical system of claim 1, wherein the texture is formed by the electrode having an arithmetic average roughness profile (Ra) between about 52 to about 64.

7. The electrosurgical system of claim 1, wherein the texture is formed in a crisscross pattern.

8. The electrosurgical system of claim 1, wherein the liquid medium is blood.

9. The electrosurgical system of claim 1, further comprising a dilator positioned near a distal end of the crossing device, proximate from the electrode.

10. The electrosurgical system of claim 1, wherein the crossing device is a wire.

11. The electrosurgical system of claim 10, further comprising an electrically insulative layer extending over the wire except at the electrode.

12. The electrosurgical system of claim 1, wherein the crossing device is a needle.

13. An electrosurgical system for puncturing tissue, the electrosurgical system comprising:
an electrosurgical generator configured to generate radiofrequency (RF) energy; and
a crossing device connected to the electrosurgical generator, the crossing device including an electrode positioned at a distal tip of the crossing device, wherein the electrode has a helical coil that is configured to form and hold a gaseous insulation layer that is formed when the crossing device is positioned in an electrically conductive liquid medium and is energized by the electrosurgical generator.

14. The electrosurgical system of claim 13, wherein the helical coil is wrapped around a central elongate member.

15. The electrosurgical system of claim 14, wherein a diameter of the helical coil is between one fourteenth and one tenth of a diameter of the central elongate member.

16. The electrosurgical system of claim 15, wherein the central elongate member includes an exterior electrically insulating layer extending proximal from the electrode.

17. The electrosurgical system of claim 13, wherein the liquid medium is blood.

18. A method of operating an electrosurgical system, the method comprising:
positioning a crossing member, wherein the crossing member includes a crossing device;
energizing an electrode of the crossing device using an electrosurgical generator;
forming and coalescing gaseous bubbles on the electrode; and
vaporizing tissue with the energized electrode.

19. The method of claim 18, wherein the crossing member is positioned near to the tissue but not in contact with the tissue prior to energizing the electrode.

20. The method of claim 18, wherein the crossing member is positioned in contact with the tissue prior to energizing the electrode.
